(19) United States
(12) Patent Application Publication
(10) Pub. No.: US 2024/0123002 A1
(43) Pub. Date: Apr. 18, 2024

(54) MICROBIOME IDENTIFICATION AND BACTERIOPHAGE FORMULATIONS

(71) Applicants: Nathan Brown, Corvallis, OR (US); Natalise Kalea Robinson, Hillsborough, CA (US)

(72) Inventors: Nathan Brown, Corvallis, OR (US); Natalise Kalea Robinson, Hillsborough, CA (US)

(21) Appl. No.: 18/466,675

(22) Filed: Sep. 13, 2023

Related U.S. Application Data
(63) Continuation of application No. PCT/US2022/021021, filed on Mar. 18, 2022.
(60) Provisional application No. 63/163,375, filed on Mar. 19, 2021.

(51) Int. Cl. A61K 35/76; A61P 31/04; C12Q 1/6806; C12Q 1/6869; C12Q 1/689

(52) U.S. Cl. CPC A61K 35/76; A61P 31/04; C12Q 1/6806; C12Q 1/6869; C12Q 1/689

(57) ABSTRACT

Provided herein are methods for treating a health condition of a subject. The method may comprise detection of a microorganism in a sample from the subject and, subsequent to identification of the microorganism, generating a bacteriophage mixture comprising at least one bacteriophage capable of lysing the microorganism. One or more processes described herein may comprise shotgun metagenomic sequencing.

### CROSS-REFERENCE

This application is a continuation application of International Patent Application No. PCT/US2022/021021, filed Mar. 18, 2022, which claims the benefit of U.S. Provisional Patent Application No. 63/163,375, filed Mar. 19, 2021, each of which is incorporated by reference herein in its entirety.

### BACKGROUND

Biological samples obtained from a subject may comprise a variety of different microorganisms, which altogether may form a microbiome of the subject or a body (e.g., skin, gut) of the subject. A microbiome may be useful in understanding one or more health parameters of the subject, or in identifying one or more pathologies.

The skin microbiome may comprise a community of microorganisms that live in and on the skin of a subject. Methods of measuring and analyzing the skin microbiome may include biological analysis, such as DNA sequencing. However, current methods of measuring the skin microbiome may introduce bias during sampling, measurement, and analysis, which can lead to inaccurate diagnosis, treatment and monitoring.

### SUMMARY

Recognized herein is a need for improved methods and systems for measuring a microbiome in a body (e.g., skin) of a subject. The present disclosure provides for systems and methods for detecting a population of microorganisms in a biological sample obtained from the subject and methods for treating one or more conditions (e.g., skin condition) of the subject.

In an aspect, provided herein is a method of treating a skin condition of a subject, comprising: (a) detecting a population of microorganisms in a skin sample obtained from the subject, wherein the detecting comprises performing shotgun metagenomic sequencing of nucleic acid molecules extracted from the population of microorganisms, thereby generating a set of sequence reads, and processing the set of sequence reads; and (b) administering to the subject a bacteriophage or fungal virus formulation comprising at least one virus capable of lysing a microorganism of the population of microorganisms.

In some embodiments, the set of sequence reads consists of between 0.1M-100M sequence reads. In some embodiments, the population of microorganisms comprises bacteria or fungi. In some embodiments, the method further comprises detecting one or more mites in the skin sample obtained from the subject. In some embodiments, the population of microorganisms comprises one or more microorganisms associated with acne. In some embodiments, the population of microorganisms comprises one or more microorganisms associated with inflammation, redness, eczema, rosacea, enlarged hair follicle pore size, rough skin texture, increased trans-epidermal water loss, skin discoloration, or disproportionate elasticity of a stratum corneum and underlying dermis. In some embodiments, the population of microorganisms comprises one or more microorganisms associated with aged skin. In some embodiments, the population of microorganisms comprises a Cutibacterium acnes bacterium. In some embodiments, the population of microorganisms comprises a Staphylococcus aureus bacterium. In some embodiments, the population of microorganisms comprises a Corynebacterium bacterium. In some embodiments, the population of microorganisms comprises a Malassezia yeast. In some embodiments, the detecting further comprises (1) isolating DNA from the skin sample, and (2) removing human DNA from the isolated DNA. In some embodiments, the method further comprises adding an internal DNA standard to the isolated DNA. In some embodiments, the internal DNA standard comprises synthetic DNA. In some embodiments, the processing comprises eliminating one or more background sequence reads from the set of sequence reads, wherein the background sequence reads are identified by shotgun genomic sequencing of DNA isolated from a negative control sample. In some embodiments, the processing comprises normalizing counts of the set of sequence reads to generate a set of normalized sequence reads. In some embodiments, the processing comprises clustering of the set of normalized sequence reads. In some embodiments, the processing comprises comparison of the set of normalized sequence reads to a database. In some embodiments, the database comprises sequences of a plurality of microorganisms isolated from other skin samples from other subjects. In some embodiments, the database comprises sequences of mobile genetic elements. In some embodiments, the mobile genetic elements comprise one or more members of the group consisting of plasmids, prophages, and transposons. In some embodiments, the database comprises genomic sequences of microorganisms isolated from other skin samples from other subjects. In some embodiments, the mobile genetic elements comprise one or more members of the group consisting of plasmids, prophages, and transposons. In some embodiments, the bacteriophage or fungal virus formulation does not comprise a lysogenic bacteriophage. In some embodiments, the bacteriophage or fungal virus formulation comprises a first bacteriophage and a second bacteriophage. In some embodiments, the first bacteriophage and the second bacteriophage are capable of lysing a microorganism of the population of microorganisms. In some embodiments, the first bacteriophage and the second bacteriophage have a different host range. In some embodiments, the different host range comprises at least two C. acnes strains. In some embodiments, the different host range comprises at least one C. acnes strain and at least one C. namnatense strain. In some embodiments, the different host range does not comprise a C. granulosum strain. In some embodiments, the first bacteriophage and second bacteriophage individually or collectively prevent resistance or regrowth of the microorganism in vitro. In some embodiments, the first bacteriophage and the second bacteriophage are lytic bacteriophage. In some embodiments, the bacteriophage or fungal virus formulation is part of a cosmetic formulation. In some embodiments, the cosmetic formulation comprises one or more liposomes comprising the first bacteriophage and the second bacteriophage. In some embodiments, the cosmetic formulation further comprises an anti-aging component.

In another aspect, provided herein is a method for determining a microbiome type, comprising: (a) combining DNA isolated from a skin sample of a subject with an internal DNA standard to form a DNA mixture, (b) performing shotgun metagenomic sequencing of the DNA mixture to generate a set of sequence reads; and (c) processing the set of sequence reads to determine the microbiome type.

In some embodiments, the subject has acne. In some embodiments, the subject has one or more skin conditions selected from the group consisting of redness, eczema, rosacea, and aged skin. In some embodiments, the DNA isolated from the skin sample comprises less than 10% human DNA. In some embodiments, the internal DNA standard is a synthetic internal DNA standard. In some embodiments, the processing comprises transforming the set of sequence reads to generate transformed sequence reads. In some embodiments, the method further comprises clustering the transformed sequence reads. In some embodiments, the processing comprises normalizing a count of a subset of the set of sequence reads to another count of a set of sequence reads from the internal DNA standard. In some embodiments, the processing comprises comparing the plurality of sequence reads to a set of sequence reads of a negative control sample, wherein the negative control sample is opened to air briefly along with the skin sample and is processed in parallel as the skin sample. In some embodiments, the processing comprises detecting one or more subtypes of Cutibacterium acnes. In some embodiments, the processing comprises detecting one or more subtypes of Staphylococcus aureus. In some embodiments, the processing comprises detecting one or more subtypes of Corynebacterium.

In another aspect, disclosed herein is a method, comprising: (a) contacting (i) a sample from an environmental source with (ii) one or more cultured microorganisms from a skin sample; and (b) isolating a lytic bacteriophage capable of lysing the one or more cultured microorganisms; and (c) sequencing a nucleic acid isolated from the lytic bacteriophage.

In some embodiments, the environmental source is sewage or a soil suspension. In some embodiments, the method further comprises identifying the one or more cultured microorganisms by a method comprising DNA sequencing. In some embodiments, the one or more cultured microorganisms comprise a Cutibacterium acnes strain. In some embodiments, the one or more cultured microorganisms comprise a Staphylococcus aureus strain. In some embodiments, the one or more cultured microorganisms comprise a Corynebacterium strain. In some embodiments, the skin sample comprises a skin swab from skin affected by acne, inflammation, redness, eczema, rosacea, or aging. In some embodiments, the method further comprises determining a host range of the lytic bacteriophage. In some embodiments, the host range does not include a helpful bacteria associated with a low risk of a skin condition. In some embodiments, the method further comprises building a bacteriophage library comprising the lytic bacteriophage. In some embodiments, the bacteriophage library comprises a plurality of lytic bacteriophages capable of lysing the one or more cultured microorganisms.

In another aspect, disclosed herein is a method of selecting bacteriophages for inclusion in a bacteriophage formulation comprising, (a) preparing a mixture of a first bacteriophage and a second bacteriophage; (b) contacting the mixture with a cultured microorganism, and (c) selecting the first bacteriophage and the second bacteriophage for inclusion in the bacteriophage formulation if the cultured microorganism is incapable of regrowth after the contacting, wherein the cultured microorganism comprises a Cutibacterium bacterium, a Staphylococcus bacterium, or a Corynebacterium bacterium.

In some embodiments, the first bacteriophage and the second bacteriophage have different host ranges. In some embodiments, the first bacteriophage and the second bacteriophage are non-lysogenic bacteriophages. In some embodiments, the method further comprises (d) preparing a cosmetic formulation comprising the first bacteriophage and the second bacteriophage. In some embodiments, the cosmetic formulation comprises liposomes. In some embodiments, the first bacteriophage and the second bacteriophage can lyse the cultured microorganism after storage for at least two months in the cosmetic formulation.

In another aspect, provided herein is a method, comprising: (a) using shotgun metagenomic sequencing to identify a host bacterium of a subject (b) upon identifying the host bacterium, generating a bacteriophage mixture comprising a first bacteriophage, a second bacteriophage, and a stabilizing buffer, wherein the first bacteriophage and the second bacteriophage are both capable of infecting the host bacterium.

In some embodiments, the host bacterium is a Cutibacterium acnes bacterium. In some embodiments, the first bacteriophage and the second bacterium do not infect helpful strains of Cutibacterium acnes. In some embodiments, the host bacterium is a Staphylococcus aureus bacterium. In some embodiments, the first bacteriophage and the second bacteriophage do not infect Staphylococcus epidermidis. In some embodiments, the host bacterium is a Corynebacterium bacterium. In some embodiments, the first bacteriophage and the second bacterium do not infect helpful strains of Corynebacterium. In some embodiments, the first bacteriophage and the second bacteriophage are encapsulated in one or more liposomes. In some embodiments, the bacteriophage mixture comprises a retinoid (vitamin A derivatives), niacinamide (vitamin B3), ascorbic acid (vitamin C), acetate or tocopherols (vitamin E derivatives), skin-active peptides, plant growth factors such as kinetin, or ubiquinone (coenzyme Q10). In some embodiments, the first bacteriophage or the second bacteriophage specifically infects one or more members of the group consisting of a Cutibacterium acnes bacterium, a Staphylococcus aureus bacterium, and a Corynebacterium bacterium.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows example data from a bacteriophage host range assay.

FIGS. 2A-2D show example data of bacterial growth in response to contacting with one or more bacteriophages.

FIG. 3 shows a computer system that is programmed or otherwise configured to implement methods provided herein.

FIG. 4 shows another set of example data from a bacteriophage host range assay.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein, in some aspects, are systems, compositions and methods for determining a microbiome type from skin samples from a subject. The determining of the microbiome type may comprise detecting a population of microorganisms in a skin sample obtained from the subject. Such detection can be useful in diagnosing or treating a skin condition (e.g., acne, inflammation, aging) of the subject and/or determining how a population of microorganisms or subpopulations therein influence or affect the skin condition. In some instances, a treatment may be administered to the subject based on the detected microbiome type. Such a method of treatment may comprise administering to the subject a formulation that can be used to selectively lyse or inhibit growth of a particular microorganism (e.g., bacteria) in the population of microorganisms. In some aspects, the formulation may comprise at least one bacteriophage capable of lysing a microorganism of the population of microorganisms. Such methods and compositions may be useful in personalized treatment of one or more skin conditions.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “sample,” as used herein, generally refers to a biological sample of a subject. The sample may be a tissue sample, such as a biopsy, core biopsy, needle aspirate, or fine needle aspirate. The sample may be a swab (e.g., from the mouth, skin or other external-facing body). The sample may be a skin sample, e.g., obtained from a swab, pore strip, skin aspirate, etc. The sample may be a fluid sample, such as a blood sample, urine sample, or saliva sample. The sample may be a cheek swab. The sample may be a plasma or serum sample. The sample may be a cell-free sample. The sample may be or comprise a viral sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears. The biological sample may comprise any number of macromolecules, for example, cellular macromolecules. The sample may be a cell sample. The sample can include one or more cells or viruses. The sample can include one or more microorganisms. The biological sample may be a nucleic acid sample or protein sample. The biological sample may also be a carbohydrate sample or a lipid sample. The biological sample may be derived from another sample.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer), or a pre-disposition to the disease, an infection, a health condition, and/or an individual that is in need of therapy or suspected of needing therapy. A subject can be a patient.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA, complementary DNA, etc.). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

Microbiome Type Identification and Bacteriophage Formulations

In an aspect, provided herein are methods for determining a microbiome type or measuring a microbiome of a body (e.g., skin, gut) of a subject. A method of the present disclosure may comprise detecting a population of microorganisms or determining a microbiome type in a sample (e.g., skin sample) obtained from the subject. In some instances, the detecting comprises performing metagenomic sequencing (e.g., shotgun metagenomic sequencing) of nucleic acid molecules extracted from the population of microorganisms. Such a method of detection may comprise generating a set or plurality of sequence reads and processing the set or plurality of sequence reads. In some aspects of the present disclosure, the method may additionally comprise administering a bacteriophage formulation to the subject, in which the bacteriophage formulation comprises at least one bacteriophage capable of lysing a microorganism of the population of microorganisms. The methods provided herein may be useful in identifying, diagnosing, or treating a skin condition (e.g., acne, inflammation, redness, eczema, rosacea, aging, etc.) of the subject.

Skin microbiome: In some aspects, the present disclosure provides methods for analyzing skin microbiome types and determining one or more microorganisms on a skin sample. The skin microbiome may comprise a population of microorganisms that live in and on the skin. The population of microorganisms may comprise a same type of microorganisms, or the population of microorganisms may comprise different types of microorganisms. For example, the population of microorganisms may comprise one or more bacteria, yeast, fungi, protists, viruses, or a combination thereof. In some instances, the population of microorganisms may comprise one or more mites.

Skin sample: The skin sample may be obtained from the subject using one or more approaches. In some instances, the skin sample is obtained using a swab. The swab may comprise any useful shape or material. For example the swab may be rod-shaped and comprise a first end comprising the specimen-collecting area. The specimen-collecting area may be any useful shape, such as round, triangular, pyramidal, rectangular, rhomboidal, pentagonal, hexagonal, heptagonal, octagonal, etc. The swab may comprise any useful material, such as a synthetic material (e.g., polymer, such as nylon, rayon, Dacron), or a naturally occurring material (e.g., cotton, bamboo, etc). In some instances, the swab is a synthetic flocked swab. In some instances, the skin sample is obtained using a tape strip. For example, the tape strip may be a pore strip, which may be used to extract the contents from a pore or pilosebaceous unit. In some instances, the skin sample is obtained using a skin biopsy.

Skin sample processing: The skin sample may be processed prior to detection of the one or more microorganisms. Such processing may comprise, for example, extraction or isolation of one or more nucleic acid molecules (e.g., DNA, RNA) from the sample and optionally, removing contaminating specimens from the sample. In some instances, contaminating nucleic acid molecules (e.g., DNA, RNA) may be removed from the sample. For example, the DNA from the subject or host (e.g., a human) may be removed from the skin sample or separated from the DNA of other organisms in the skin sample. Alternatively or in addition to, the microbial DNA from the skin sample may be enriched or purified. Such an enrichment of microbial DNA or host DNA depletion may comprise techniques such as osmotic lysis, propidium monoazide treatment, or other approaches in order to lyse human cells and/or degrade mammalian or human DNA. Accordingly, the processed sample may include minimal human DNA, e.g., less than 50%, less than 40%, less than 30%, less than 20%, less than 10%, less than 5%, less than 1% or less of human DNA.

In some instances, an internal standard may be added to the isolated or extracted nucleic acid molecules. Inclusion of an internal standard (e.g., DNA standard) may be performed by spiking known DNA sequences in the sample (or processed sample, e.g., the isolated or extracted DNA). In some instances, adding an internal standard may be useful in adjusting relative sequence read counts obtained from sequencing or for normalizing counts of sequence reads, thereby improving the quantitative accuracy of the sequencing. The internal standard may be a synthetic internal DNA standard, or the internal standard may comprise naturally occurring DNA molecules. The internal DNA standard may comprise metagenome sequins, such as those described in Hardwick, et al. 2018. “Synthetic Microbe Communities Provide Internal Reference Standards for Metagenome Sequencing and Analysis.” Nature Communications 9 (1): 3096, which is incorporated by reference herein in its entirety.

The inclusion of control samples may also be used for improving accuracy of determining microbiome subtypes. In one example, a negative control sample, e.g., a swab that is exposed to the environment or air, may be included for microbiome measurement or analysis. In such an example, the skin sample may be collected on a first set of swabs, and a negative control set of swabs which are exposed to the air and not to the subject's skin may be included for further processing, as described herein, and analysis, e.g., via sequencing to generate a set of negative control sequence reads. The negative control sequence reads, or a portion thereof, may be subtracted or removed from the sequence reads (e.g., in silico) of the skin sample. Such a technique may be useful in removing background nucleic acid contamination (e.g., removing environmental or airborne bacterial DNA).

Further processes to prevent contamination or remove contaminants from the skin samples may be implemented. For instance, sterile handling techniques may be practiced, e.g., processing samples in a sterilized laminar flow hood, sterilization of tools or reagents, treatment of sterilization of tools or agents with enzymes (e.g., RNAse or DNAse) to remove contaminating nucleic acid molecules, etc. As will be appreciated, any number of processing, decontaminating, and/or enrichment techniques may be used to optimize accuracy of sample readout (e.g., via sequencing reads). One or more of the processing, decontaminating and/or enrichment techniques may improve sample readout by removing or reducing bias during sample analysis, e.g., via sequencing, clustering of sequence reads into skin microbiome types, and identification of one or more microorganisms. Accordingly, accuracy of identification of skin microbiome types may be improved using such sample processing.

Measurement of the skin microbiome: Microbiome type determination or identification of the one or more microorganisms on the skin sample may be performed using a DNA sequencing (e.g., high throughput DNA sequencing) method. Sequencing may comprise 16S rRNA sequencing or shotgun metagenomic sequencing. In some instances, shotgun metagenomic sequencing may be performed on the skin samples, which may enable accurate identification of skin microbiome types. Shotgun metagenomic sequencing may be useful in comprehensively sampling all genes present in a complex sample (e.g., comprising nucleic acid molecules from multiple microorganism types), thereby enabling detection or identification of one or more (or all) microorganisms present in the sample. In some instances, the use of shotgun metagenomic sequencing may obviate the use of 16S rRNA sequencing, which may have limited taxonomic resolution or introduce bias into skin microbiome analysis, e.g., due to omission of microorganisms or viruses lacking a 16S rRNA gene or omission of microorganisms (e.g., bacterial taxa) from primer design. In other instances, the use of shotgun metagenomic sequencing may supplement the use of 16S rRNA sequencing. Shotgun metagenomic sequencing may be useful in detecting non-bacterial microorganisms, such as, for example, yeast (e.g., Malassezia yeast), mites (e.g., Demodex), etc. Beneficially, detection of non-bacterial microorganisms may allow for more accurate profiling of the skin sample and correlating different microorganism types with particular skin conditions (e.g., folliculitis, eczema, redness, acne, rosacea, aged skin, etc.) or skin appearance.

Shotgun metagenomic sequencing: In some instances, shotgun metagenomic sequencing may be used to generate sequence reads from one or more organisms of the sample. Shotgun metagenomic sequencing can be useful in resolving strain-level variants that may not be resolvable from 16S rRNA sequencing. For example, shotgun metagenomic sequencing may be useful in determining sequences of microbial accessory genomes that can be undetectable from 16S rRNA sequencing. Such accessory genomes can include, in non-limiting examples, genetic elements that are strain-specific or not essential for survival of the microbe. In some instances, the shotgun metagenomic sequencing may be used to resolve or detect mobile genetic elements, such as plasmids, prophages, or transposons. The mobile genetic elements may comprise different accessory genes, which may confer properties such as antibiotic resistance, virulence, or metabolic pathways. Determining the strain-level variation of a sample may aid in determining how one or more microorganisms (e.g., bacteria such as C. acnes or S. aureus, yeast, protists, etc.) or the genetic elements of the microorganisms, contribute or influence a skin condition (e.g., acne, aging, inflammation). Alternatively or in addition to, determining the strain-level variation of a sample may allow for determination of microorganism susceptibility (e.g., to a small molecule treatment, bacteriophage, etc.) and virulence factors. Accordingly, the information obtained from shotgun metagenomic sequencing may be used to design a formulation (e.g., bacteriophage formulation) known to target a particular microorganism (such as one found on the skin sample) in a diversely populated sample.

Shotgun metagenomic sequencing may also be useful in generating sufficient sequencing depth to detect rarer microorganismal or viral variants or strains. For example, shotgun metagenomic sequencing may capture strain-level variation in bacteriophage receptor genes that may be indicative of bacteriophage susceptibility and virulence factors in skin bacteria. Accordingly, any useful sequencing depth may be obtained from the shotgun metagenomic sequencing. For example, the sequence reads generated from a skin sample may comprise at least 0.1 million (M), at least 1 M, at least 5 M, at least 10 M, at least 20 M, at least 30 M, at least 40 M, at least 50 M, at least 60 M, at least 70 M, at least 80 M, at least 90 M, at least 100 M, at least 200M or more sequence reads. In some instances, the sequence reads generated from the skin sample may comprise at most 200 M, at most 100 M, at most 90 M, at most 80 M, at most 70 M, at most 60 M, at most 50 M, at most 40 M, at most 30 M, at most 30 M, at most 10 M, at most 5 M, at most 1 M, at most 0.1 M or fewer sequence reads. A range of sequence reads may be generated from a skin sample, e.g., between about 1 M to about 100 M sequence reads.

Analysis of metagenomic data: Following shotgun metagenomic sequencing, the sequence reads may be further processed or analyzed (e.g., in silico). Such processing or analysis may include, in non-limiting examples: filtering, assembly, binning, gene prediction, etc. and may be performed using one or more processors. In an example, the sequence reads may be pre-filtered, e.g., via removal of redundant sequences, low-quality sequences, contaminating sequences, etc. In some instances, the sequence reads may be assembled to determine the origin (e.g., the microorganism, the subject, etc.) from which a sequence read originates. Such assembly can be performed using one or more assembly programs, e.g., Phrap, Celera Assembler, Velvet Assembler, etc. Alternatively or in addition to, the assembly may be performed by generating de Brujin graphs and assembling the reads. One or more sequence reads generated from the sequencing may be compared to a reference genome, e.g., for assembly, binning, gene prediction, etc. The assembled sequences or contigs may be further annotated for coding regions. Such annotation may comprise identification of genes based upon sequence homology with known gene sequences (e.g., homology with publicly available sequence databases, e.g., BLAST, RefSeq). Any useful program may be implemented for the gene prediction, e.g., MEGAN, GeneMark, GLIMME, MetaProdigal, FragGeneScan, MetaGeneMark, SNAP, EuGene, and the like. In some instances, custom algorithms may be generated for assembly, annotation, or any other processing operation. Alternatively, assembly is not performed, and the sequences may be further processed, e.g., for taxonomic profiling, functional analyses, etc.

In some instances, the assembled sequences or contigs (or the annotated sequences) are binned to associate a sequence with an organism. Such binning may be performed using homology-based approaches (e.g., using BLAST to search for phylogenetic markers, or other programs such as Meta Genome Analyzer (MEGAN), PhymmBL, MetaPhiAn, AMPHORA, mOTUS, MetPhyler, SLIMM, etc.). Other binning algorithms include, but are not limited to: DAS Tool MetaWRAP, CheckM, AMBER, SolidBin, CARMA, Sort-iTEMS, TACOA, PhyloPythiaS, Phymm, SCIMM, Metawatt, LikelyBin, MaxBin, COCACOLA, MetaBAT2, CONCOCT, BMC3c, MetaBMF, MBCC, Canopy.

In some instances, the sequence reads, or derivatives thereof (e.g., assembled reads, assembled contigs, binned reads, etc.) may be transformed. For example, proportionality may be employed (e.g., applied to the sequence reads) to determine a proportion of the reads that are assigned to a microbial taxon. The transformation may be performed at any useful processing operation, e.g., prior to, during, or following clustering or binning of sequence reads, prior to, during, or following assembly, etc.

The sequence reads may be normalized to determine, for example, a relative count of each sequence within the sample or to compare counts of sequences across samples. Such a count normalization may be performed, for example, by including appropriate controls during sample processing, e.g., by including (e.g., via spike-in) a synthetic internal DNA standard in each sample or DNA library prior to sequencing, as is described elsewhere herein. Following sequencing, the counts of each sequence read (e.g., from the one or more microorganisms) from the sample may be normalized to the counts of the DNA standard, thereby obtaining a relative count of each sequence read. Accordingly, the relative read counts from a first sample (e.g., a first skin sample from a subject) may be compared to the relative read counts in another sample (e.g., a second skin sample from the subject or a different subject), allowing quantitative comparison of the sequence reads across samples. Beneficially, the normalization of each sequence read count may improve accuracy of quantitation of the sequence reads present within a sample or across multiple samples. Normalization of the sequence reads may occur at any useful or convenient time of the sequence data analysis, e.g., prior to clustering, prior to binning, prior to assembly, etc.

The sequence reads or derivatives thereof (e.g., the normalized sequence reads) may be clustered using any useful clustering method. For example, the clustering may comprise random forest clustering. Clustering may be performed based on common features, such as unitigs from colored compact de Brujin graphs or hashed kmers.

Taxonomic profiling of the sequence reads or derivatives thereof may be performed, e.g., to identify microbial taxa (e.g., the species of microorganisms in the sample). The taxonomic profiling may include or be a part of annotation, or the taxonomic profiling may be a distinct process. Taxonomic profiling may comprise identification of genes based upon sequence homology with known gene sequences (e.g., homology with publicly available sequence databases, e.g., BLAST, RefSeq). Any useful program may be implemented for the taxonomic, e.g., MEGAN, MGS-Fast, MG-RAST, MAGpy, DIAMOND, etc. In some instances, the program used for taxonomic profiling may comprise an open source or publicly available program, e.g., Sourmash. Alternatively or in addition to, custom sequence databases, e.g., genomic databases generated from sequencing of multiple skin samples, such as those obtained using the methods described herein, may be generated and used as reference sequences for the taxonomic profiling.

Microbiome library: Skin samples from multiple subjects may be collected and subjected to sequencing (e.g., shotgun metagenomic sequencing), and the sequencing reads may be stored in a microbiome library. The microbiome library may comprise sequences of a plurality of microorganisms that are obtained from the skin samples and may comprise, for instance, DNA sequences of bacteria, yeast, mites, viruses, etc. The sequences may comprise genomic DNA or accessory genome sequences, e.g., from plasmids, transposons, prophages, etc.

In some instances, the samples or microorganisms from the samples may also be collected and stored. For example, one or more microorganisms may be removed, isolated, or extracted from the sample, and a portion of the removed, isolated, or extracted microorganisms may be subjected to sequencing, as described herein, and a portion may be cultured (e.g., collected and grown on a plate). The cultured microorganisms may be used in further screening, e.g., to identify one or more agents capable of lysing or inhibiting growth of at least one of the cultured microorganisms. As different samples may comprise different microbiomes and species of microorganisms, each cultured sample may be maintained separately from the cultured microbiomes of other samples. Accordingly, screening for agents capable of lysing of inhibiting growth of one or more microorganisms in each of these samples may enable personalized treatments of skin conditions caused by the specific microbiome type. Alternatively or in addition to, microbiomes across samples may be mixed together and screened to identify one or more agents capable of lysing or inhibiting growth of the pooled sample.

Bacteriophage assays: Screening of the samples may be performed to determine one or more agents (e.g., small molecules, bacteriophages, or biological molecules, e.g., proteins, peptides, lipids, carbohydrates, metabolites, or combinations thereof) capable of inhibiting growth or otherwise killing (e.g., via lysing) of one or more microorganisms of the samples. In some examples, the one or more agents comprises one or more bacteriophages. For example, the one or more bacteriophages may be naturally occurring, recombinant, or synthetic bacteriophages that are capable of lysing a type of bacteria present on the skin sample. In such an example, the screening of the samples may comprise: obtaining one or more microorganisms from the sample, optionally culturing the one or more microorganisms, and exposing the one or more microorganisms to a plurality of bacteriophages to generate a microorganism-bacteriophage mixture. The growth, proliferation, or death of the microorganisms may be monitored over a duration of time, and the microorganisms may be collected, stored, and/or sequenced. Sequencing (e.g., shotgun metagenomic sequencing) may yield information on the bacteriophages that are present in or on the microorganisms and that may be capable of killing or inhibiting growth of the one or more microorganisms. Accordingly, such screening, e.g., the combination of culturing microorganisms with bacteriophages and sequencing the mixture, may be useful in determining combinations of bacteriophages to remove particular microorganism (e.g., bacterial) strains.

In some instances, the screening may comprise sequencing one or more microorganisms isolated from a sample. For example, the one or more microorganisms isolated from the sample may be (i) cultured to generate a microorganism library and (ii) sequenced (e.g., via shotgun metagenomic sequencing) to generate a library of sequence reads comprising sequences from the microorganisms present within the sample. Additionally, the cultured microorganisms may be further exposed to one or more bacteriophages, and then isolated and sequenced, as described above. The sequences corresponding to the one or more bacteriophages may be stored in a bacteriophage library. Alternatively or in addition to, the one or more bacteriophages may be stored and further screened, e.g., using a bacteriophage host range assay, to determine bacterial strains which may be susceptible to the one or more bacteriophages.

Bacteriophage source: The one or more bacteriophages may be synthetic or naturally occurring. The one or more bacteriophages may be isolated or extracted from an environmental source, including, but not limited to: sewage, soil suspensions, ocean sediment, terrestrial sub-surfaces, etc. In such instances, further processing of the environmental source may be performed to purify or enrich for the bacteriophages.

Bacteriophage host range assay: A bacteriophage host range assay may be used to determine a host or suite of host cells (e.g., bacterial cells) that are susceptible to a given bacteriophage in vitro. In some instances, the bacteriophage host range assay is performed using the one or more microorganisms isolated from the sample, or a plurality of samples, and one or more bacteriophages present in an environmental sample. For example, bacteria from one or more skin samples may be collected and optionally, a portion of the collected bacteria may be sequenced for identification of the bacteria type (e.g., bacterial species or variant). The collected bacteria may be cultured (e.g., on agar plates), distributed on a phage host range assay dish, and contacted with viral fractions from an environmental source, which may comprise one or more bacteriophages (e.g., lytic bacteriophages). Optionally, the collected bacteria may be pre-sorted or processed, such that only one strain type or class of strain types is present per dish of the phage host range assay. The bacteria-bacteriophage mixtures on the phage host range assay may optionally be cultured. The bacteriophages that grow on or within the collected and cultured bacteria and/or lyse or prevent growth of the cultured bacteria on the assay may be isolated, optionally propagated, and stored as part of a bacteriophage library. Alternatively or in addition to, the bacteriophages may be sequenced. Such sequencing data may then be used to associate each of the isolated bacteriophages with a particular host organism. Accordingly, such an assay may be useful in determining the identities and genomic sequences of bacteriophages that are capable of removing one or more microorganisms from a sample. As the identities of the one or more microorganisms are also known (e.g., from sequencing), the host range of each bacteriophage may be determined. Accordingly, for a given target bacterial strain, an appropriate bacteriophage may be selected for inclusion in a formulation designed to lyse the targeted bacterial strain.

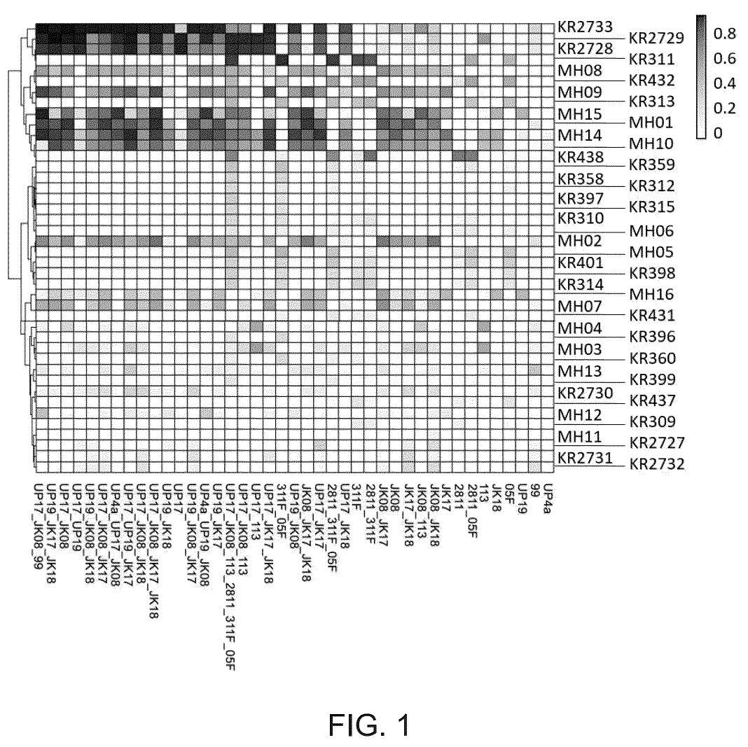

FIG. 1 illustrates an example plot of data that can be obtained from a bacteriophage host range assay. The x-axis represents bacteriophage types or combinations, and the y-axis represents bacterial strains. In such an example, each grid of the array may represent one or more microorganisms (e.g., bacteria) which are found on one or more skin samples combined with one or more bacteriophages. Each grid across the array may represent the same microorganism type (e.g., bacterial strain) or the grids may represent different microorganism types (e.g., bacterial strains). In some instances, some of the grids may represent the same microorganism type, while other grids may represent different microorganism types. Similarly, the bacteriophages represented in each grid of the array may be the same or different, or some of the grids may represent the same bacteriophages while others represent different bacteriophages. In some instances, each grid represents a different bacteria-bacteriophage combination. For example, each grid may represent growth (or lack thereof) of a single microorganism (e.g., bacteria) type (e.g., strain) and a single or combination of bacteriophage types. In the plot, the density or intensity of each grid represents higher killing or growth inhibition of the bacterial strain from the bacteriophage type or combination of bacteriophages. In some instances, one or more grids of the assay may represent more than one bacteriophage, which may be useful in determining susceptibility of a bacteria type or strain to a combination or mixture of bacteriophages. The host range data may be useful in determining combinations of bacteriophages (e.g., lytic bacteriophages) that are effective in lysing or preventing regrowth of a microbiome subtype (e.g., a target bacterial strain), which may aid in preventing bacterial resistance. In some instances, it may be beneficial to avoid lysing of certain strains of “helpful” bacteria (e.g., bacteria associated with a low risk of a skin condition, bacteria that prevent overgrowth of other bacterial types, bacteria that prevent infection from other microorganisms, certain S. epidermis bacteria, C. granulosum bacteria, etc.); accordingly, understanding of the host range may be useful in generating bacteriophage combinations that selectively avoid lysing the helpful bacterial strains.

Bacteriophage formulations: Also provided herein, in some aspects, are methods for generating bacteriophage formulations or mixtures. The bacteriophage formulations or mixtures may comprise at least one bacteriophage capable of lysing a microorganism (e.g., bacteria) or inhibiting growth of a microorganism, e.g., a microorganism found in a biological sample (e.g., skin, gut, mouth) that is obtained from a subject, and may be useful, in some instances, in treating a health condition (e.g., skin condition or disorder). A method may comprise determining or identifying a microorganism in the sample (e.g., identifying a host bacterium) and generating a bacteriophage mixture or formulation comprising at least one bacteriophage capable of infecting the microorganism. The bacteriophage mixture or formulation may be administered to the subject from which the sample was obtained. Accordingly, the methods provided herein may be useful in generating personalized formulations or mixtures to treat individual health conditions. In some instances, the bacteriophage mixture or formulation comprises at least two different bacteriophages, which may have the same or different host ranges. Such a combination of bacteriophages with a designated host range may aid in prevention of bacterial or microbial resistance.

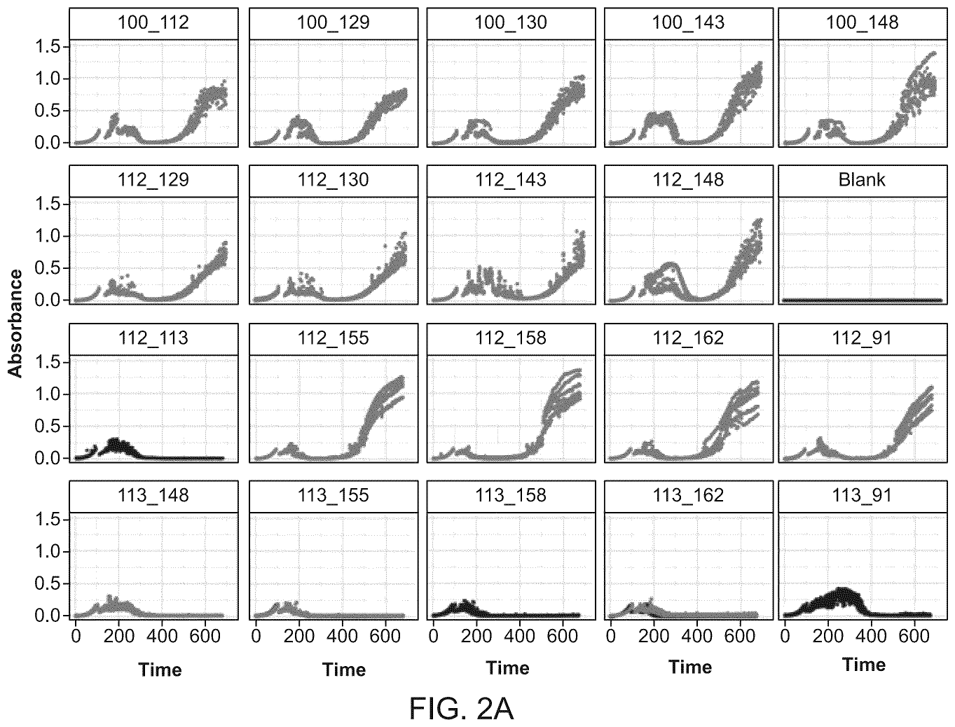

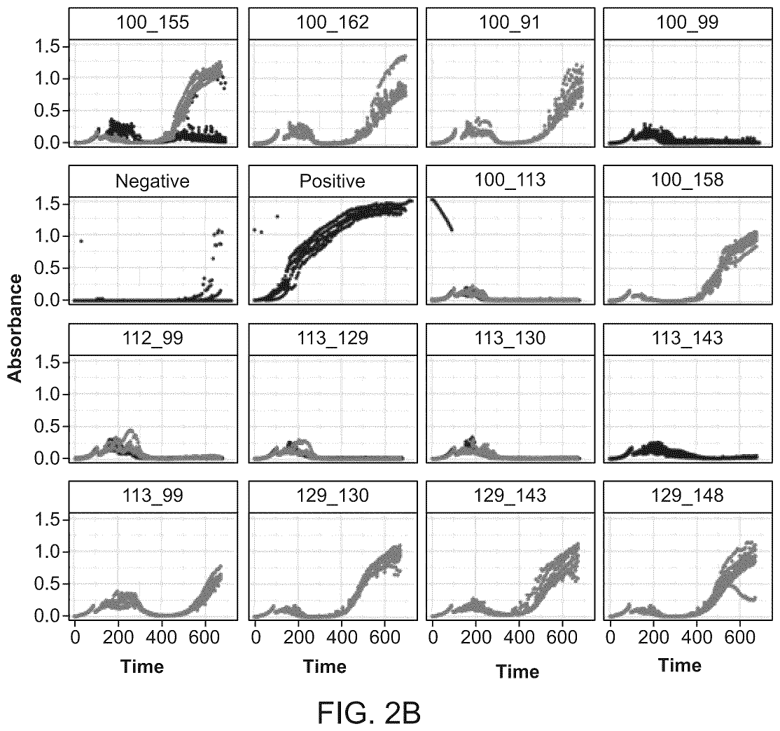

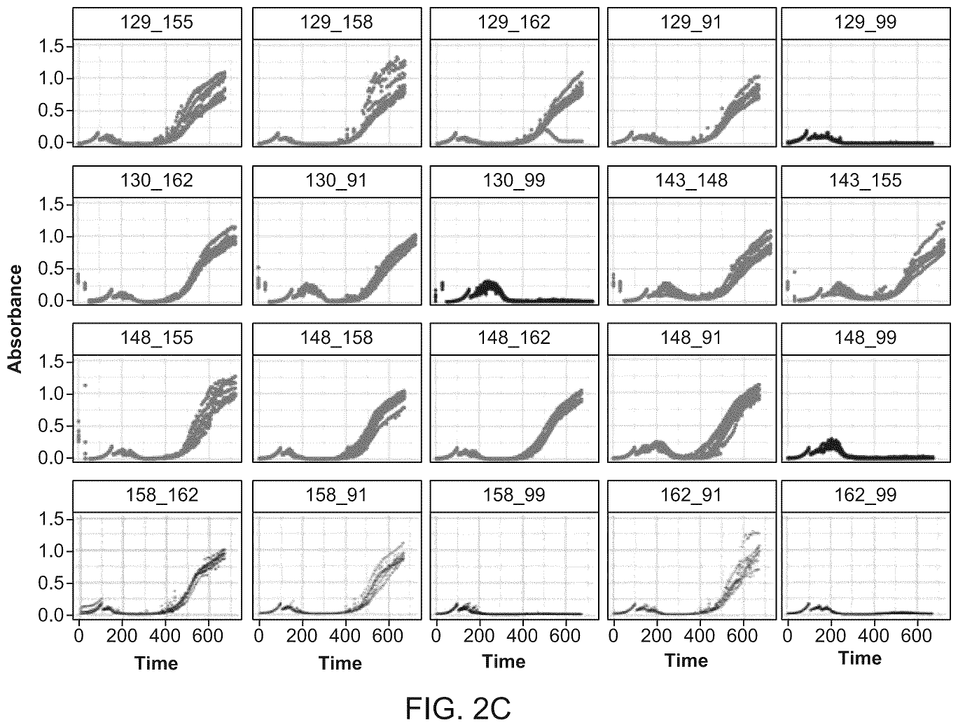

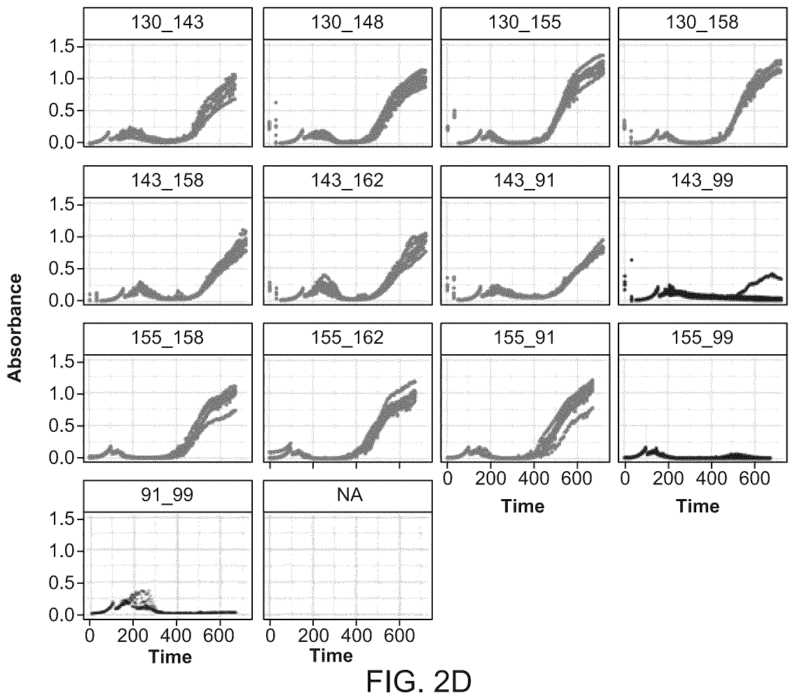

FIGS. 2A-2D illustrate example data of bacterial growth in response to infection with pairs of bacteriophages. Each panel represents a growth curve of a bacterial strain after infection with pairs of T4-like bacteriophages. The lighter-shade curves indicate bacterial regrowth after infection and the darker curves indicate little to no bacterial growth. Pairs of bacteriophages that allow little or no bacterial regrowth may be candidates for inclusion in a bacteriophage mixture or formulation for treating a skin condition associated with the bacterial strain.

Selection of bacteriophages: Bacteriophages may be selected for inclusion in a mixture or formulation based on any useful characteristic. Characteristics that may be used for selection of bacteriophages, include, in non-limiting examples, non-lysogeny or non-integration in a host genome, host range (e.g., specific to a host organism with minimal off target binding), and stability in a given formulation. In some instances, combinations of bacteriophages may be selected for a designated microbiome type or host range (e.g., for targeting a given bacterial strain such as C. acne), which in combination may aid in preventing bacterial resistance.

Formulation: The present disclosure also provides formulations for administering a bacteriophage or bacteriophage mixture, e.g., to a subject. A formulation may comprise a cosmetic formulation of skincare formulation. The formulation may comprise any useful ingredients or compositions, such as an excipient that is configured to stabilize the one or more bacteriophages (e.g., maintain a percentage of viability of the one or more bacteriophages for a duration of time, e.g., at least one week). The excipient may comprise a substance for bulking up a solid, liquid, or gel formulation comprising the one or more bacteriophages. In some cases, the substance may confer a therapeutic enhancement to the one or more bacteriophages, e.g., by enhancing solubility, decreasing or increasing dissolution, enhancing stability, increasing penetration into the skin or portion thereof (e.g., the stratum corneum), increasing bacteriophage activity, etc. In some instances, the excipient may comprise one or more liposomes or lipophilic moieties (e.g., micelles, vesicles), which may encapsulate the one or more bacteriophages. The excipient or a substance of the excipient may be used to change a property of the composition, such as the viscosity. The substance may be used to change a property of the therapeutic agent, e.g., bioavailability, absorption, hydrophilicity, hydrophobicity, pharmacokinetics, etc. The excipient may comprise a binding agent, anti-adherent agent, a coating, a disintegrant, a glidant (e.g., silica gel, talc, magnesium carbonate), a lubricant, a preservative, a sorbent, a sweetener, a vehicle, or a combination thereof. For instance, the excipient may comprise a powder, a mineral, a metal, a sugar (e.g. saccharide or polysaccharide), a sugar alcohol, a naturally occurring polymer (e.g., cellulose, methylcellulose) synthetic polymer (e.g., polyethylene glycol or polyvinylpyrrolidone), an alcohol, a thickening agent, a starch, a macromolecule (e.g., lipid, protein, carbohydrate, nucleic acid molecule), etc.

The formulation may comprise additional components for treating a skin condition (e.g., aging, acne). For example, the formulation may comprise one or more anti-aging ingredients, including but not limited to: retinoids (vitamin A derivatives), niacinamide (vitamin B3), ascorbic acid (vitamin C), skin-active peptides, proteins or peptides (e.g., collagen, hyaluronic acid, or derivatives thereof), plant growth factors, e.g., kinetin, ubiquinone (coenzyme Q10), etc. Alternatively or in addition to, the formulation may comprise one or more anti-inflammatory agents, e.g., anti-histamines, salicylates, and the like. In some instances, the formulation may comprise one or more anti-acne agents, such as benzoyl peroxide, salicylic acid, probiotics, antibiotics, antifungals, etc.

Skin conditions: The bacteriophage mixtures or formulations may be used to treat a skin condition which may be caused by or associated with a particular microbiome subtype (e.g., a particular microorganism, such as a bacterial strain, or combination of microorganisms). For example, a formulation described herein may be used to treat acne, inflammation, redness, eczema, rosacea, enlarged hair follicle pore size, rough skin texture, increased trans-epidermal water loss, skin dehydration, skin discoloration (e.g., hyperpigmentation), disproportionate elasticity of the skin or portion thereof (stratum corneum, dermis, etc.). The formulation may comprise one or more bacteriophages that are capable of lysing or inhibiting growth of one or more microorganisms that are associated with a skin condition, e.g., Cutibacterium acnes or Staphylococcus aureus, which may be associated with acne.

Microbial variants: As described herein, the skin samples may comprise one or more microorganisms associated with a skin condition (e.g., acne, eczema, redness, etc.). Accordingly, the bacteriophage mixtures and formulations may be targeted for any of the species or variants of skin microorganisms which may be associated with the skin condition. For instance, one or more bacteriophages may target a bacteria, such as a bacteria from the Actinobacteria, Firmicutes, Proteobacteria, Bacteroidetes phyla. The one or more microorganisms may comprise, in some examples, a Cutibacterium bacterium (e.g., C. acnes, C. namnatense, C. avidum), a Staphylococcus bacterium (e.g., S. aureus, S. epidermidis, S. warneri, S. pyogenes, S. mitis), a Corynebacterium bacterium, an Acinetobacter bacterium (e.g., A. johnsonii), a Pseudomonas bacteria (e.g., P. aeruginosa), other bacteria, or combinations thereof. In some instances, the one or more bacteriophages may be targeted for a fungal microorganism, including, but not limited to: yeasts, such as Candida albicans, Rhodotorula rubra, Torulopsis and Trichosporon cutaneum, dermatophytes such as Microsporum gypseum, and Trichophyton rubrum, and nondermatophyte fungi such as Rhizopus stolonifer, Trichosporon cutaneum, Fusarium, Scopulariopsis brevicaulis, Curvularia, Alternaria alternata, Paecilomyces, Aspergillus flavus and Penicillium. The one or more bacteriophages may target a combination of microorganisms; such a combination may be determined, for example, using a host range assay, as described elsewhere herein.

Kits: Also provided herein are kits for obtaining a sample (e.g., skin sample) from a subject. Such a kit may comprise, for instance, a sample collector, such as a tape patch or strip, a swab, a wipe, etc. or any other useful collector. The kit may comprise a vessel or container for holding the sample or sample collector and optionally, reagents, such as stabilization agents, buffers, preservatives, fixatives, pH balancing reagents, etc. The kit may additionally comprise instructions for sample collection, storage, transportation, treatment, etc.

Systems: In some aspect, disclosed herein are systems for determining a microbiome type from a sample (e.g., a skin sample), comprising: a sequencer configured to perform shotgun metagenomic sequencing, and one or more processors configured to process a set of sequence reads to determine a microbiome type.

### Computer Systems

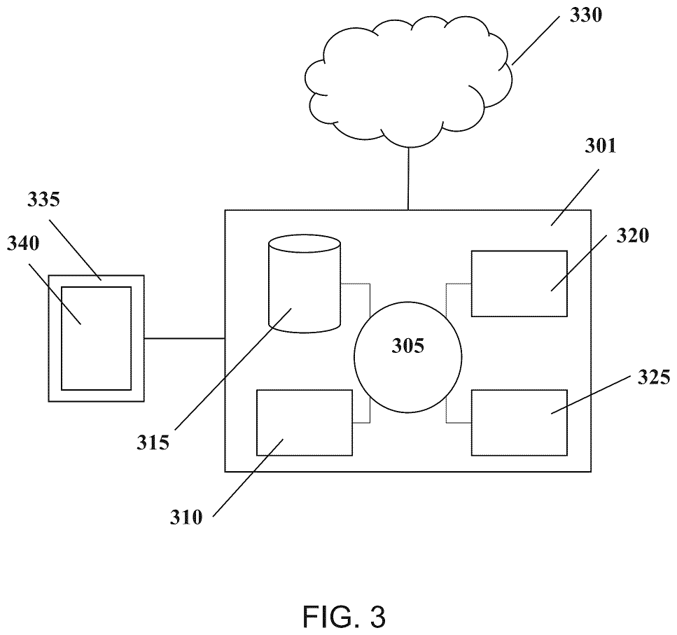

The present disclosure provides computer systems that are programmed to implement methods of the disclosure. FIG. 3 shows a computer system 301 that is programmed or otherwise configured to analyze or process sequence reads from shotgun metagenomic sequencing. The computer system 301 can regulate various aspects of processing of sequence reads of the present disclosure, such as, for example, performing clustering analysis, performing transformations, normalization of sequence reads to a standard or negative control, etc. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, sequence read alignment, binning, clustering, normalization to a standard or control, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, process sequence reads, e.g., alignment, binning, assembly, normalization, clustering, transformation, taxonomic profiling, etc.

### EXAMPLES

### Prophetic Example 1—Phage Host Range Assay for Identifying Bacteriophages that Infect C. Acnes

A bacteriophage host range assay may be used to determine a host or suite of host cells (e.g., bacterial cells) that are susceptible to a given bacteriophage in vitro. In some examples, it may be useful to identify bacteriophages that infect C. acnes bacteria. To do so, C. acnes strains may be cultured, distributed on a phage host range assay plate or plurality of plates, and contacted with bacteriophages. The bacteria-bacteriophage mixtures (e.g., from each assay of an array of assays) on the phage host range assay may optionally be cultured or incubated. Sequencing (e.g., metagenomic shotgun sequencing) may be performed on the bacteria-bacteriophage mixtures to determine the identities of the bacteriophages and/or bacteria present in each of the mixtures.

Sequencing (e.g., shotgun metagenomic sequencing) of DNA extracted from bacteriophage plaques on a host range assay plate can be used to identify one or more bacteriophages. Sequencing (e.g., shotgun metagenomic sequencing) of DNA extracted from bacteria growing on the plate can be used to identify the C. acnes strain. In some examples, DNA is extracted from both the bacteriophage plaques and the bacteria growing on the plate. In some instances, it may be beneficial to include an internal DNA standard, e.g., as a positive control or for normalizing sequence counts. For example, a DNA standard of known sequence may be spiked into a sample (e.g., a sample containing DNA extracted from the bacteriophages or bacteria) prior to sequencing. Subsequent to sequencing, the presence of positive control reads may be helpful in verifying that sequencing was performed accurately. In some instances, the internal DNA standard may be used to normalize or quantitate the sequencing counts. For example, the internal DNA standard may be spiked into a sample at a known concentration, and subsequent sequencing read counts may be normalized based on the starting known concentration to determine a relative or absolute quantity of each of the obtained sequences.

The sequencing data (e.g., sequence reads) may be further processed or analyzed, as described herein, e.g., to identify genetic elements of a bacteriophage associated with a host-range pattern. For example, one or more clustering analyses may be performed to determine sequence segments that are common to bacteriophages capable of infecting a given C. acnes strain. Accordingly, the determined sequence segments may be useful in identifying bacteriophages or bacteriophage types that are capable of lysing C. acnes.

### Prophetic Example 2—Skin Microbiome Type or Subtype Determination

The methods, systems, and kits described herein may be useful in determining a microbiome type or subtype from a subject and administering a composition comprising a bacteriophage that is capable of treating a condition of the subject.

In one example, a skin sample (e.g., using a kit described herein) may be obtained from a subject. The subject may have or be suspected of having a skin condition (e.g., acne). The skin sample may be further processed, e.g., to extract DNA from the skin sample. An internal standard may be added to the extracted DNA. The extracted DNA may be subjected to sequencing (e.g., metagenomic shotgun sequencing), which may be used to determine a microbiome type or subtype of the subject. For example, the sequenced DNA may indicate the presence of one or more C. acnes bacteria or other bacterial strains that may be associated with the skin condition (e.g., acne). The relative abundance of each bacteria may be determined with reference to sequence reads from the internal standard. Subsequently, a composition comprising one or more bacteriophages may be administered to the subject. The one or more bacteriophages may comprise a bacteriophage capable of infecting and lysing a bacterial strain present in the skin sample; for instance, for a skin sample comprising one or more C. acnes bacteria, one or more bacteriophages capable of lysing the C. acnes bacteria may be administered to the subject, which may be useful in treating the skin condition (e.g., acne). In some examples, the composition may comprise two or more bacteriophages that can lyse the same C. acnes strain, which may help reduce bacterial resistance. In some examples, the composition may comprise two or more bacteriophages that can lyse different C. acnes strains. In some examples, a bacteriophage cocktail may be administered to target one or more bacterial strains associated with the skin condition. The one or more bacteriophages may specifically target bacterial strains associated with the skin condition (e.g., acne) and not other bacteria that are not associated with the skin condition (e.g., C. granulosum). Administration of such compositions comprising the one or more bacteriophages to the subject may be useful in treating the skin condition.

### Example 3—Bacteriophage Host Range Assay for C. acnes, C. Namnatense, and C. granulosum

As described herein, a bacteriophage host range assay may be used to determine a host or suite of host cells (e.g., bacterial cells) that are susceptible to a given bacteriophage in vitro.

An example of a bacteriophage host-range assay protocol is provided as follows:
1. Prepare an agar-overlay petri dish by combining 10 mL of molten top agar and less than 500 μL of the putative host bacterium and pouring on top of a solid base of 1.5% agar media in a 12 cm-square petri dish.
2. Serially dilute a phage stock in SM buffer.
3. Pipette 5-10 microliters (μL) of the 10 mL and then 10-4 dilution (or some other chosen dilutions) of the phage stock in an organized grid onto the agar-overlay petri dish.
4. Repeat steps 2-3 with different phage stocks.
5. Allow the 10 μL phage droplets from each phage stock dilution to absorb into the top agar.
6. Place the agar-overlay plates into the incubator under appropriate growth conditions for the host bacterium.
7. After the bacteria have formed a visible lawn during incubation, evaluate the plates for the development of plaque formation where the phage dilutions were pipetted onto the agar-overlay petri dish.
8. If plaque formation is visible on one or both of the phage spots, then score the phage host range accordingly.

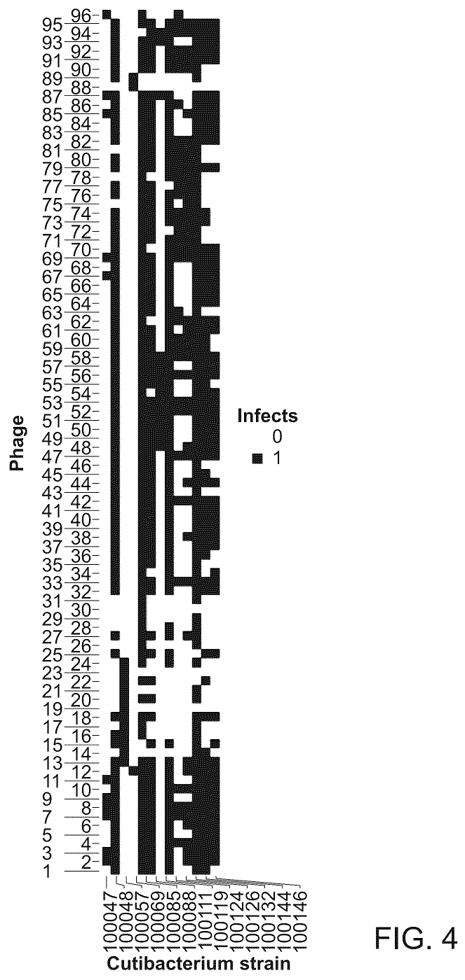

FIG. 4 illustrates example data of a bacteriophage host range assay generated using the abovementioned protocol. The y-axis represents 96 different bacteriophages, and the x-axis represents thirteen different bacterial strains. The bacterial strains of the x-axis represent, from left to right, strain numbers 100047 (a C. namnatense strain), 100048 (a C. acnes subsp defendens strain), 100057 (a C. avidum strain), 100069 (a C. granulosum strain), 100085 (a C. acnes strain), 100088 (a C. acnes strain), 100111 (a C. acnes strain), 100119 (a C. acnes strain), 100124 (a C. acnes strain), 100126 (a C. acnes strain), 100132 (a C. acnes strain), 100144 (a C. acnes strain), and 100146 (a C. acnes strain). Each grid of the array represents a single assay of a bacterial strain (x-axis) that is contacted with a bacteriophage (y-axis). In the plot, the density or intensity of each grid represents killing or growth inhibition of the bacterial strain from the bacteriophage type. The white squares indicates that the bacteriophage do not infect the bacterial strain. The black squares indicate that the bacteriophages do infect the bacterial strain.

As can be observed in FIG. 4, all except for 11 of the bacteriophages do not infect the C. namnatense strain (first column), and all except for 3 of the bacteriophages do not infect the C. granulosum strain (fourth column). Many of the phages infect multiple C. acnes strains. The bacteriophages having useful properties (e.g., infecting most or all C. acnes strains and C. namnatense but not C. granulosum) may be selected for inclusion in a formulation, e.g., to treat acne.

Overall, the host range data may be useful in determining bacteriophages that are effective in lysing or preventing regrowth of particular bacterial strains. In addition, the host range data may be useful in selecting combinations of bacteriophages for lysing or preventing regrowth of one or more bacterial strains. For example, the host range data may be useful in determining two or more bacteriophages that can lyse the same or different C. acnes strains (for example bacteriophage 1-13 are all capable of lysing C. acnes strain 100048); such a combination of bacteriophages may be useful to prevent the emergence of variant strains that are resistant to one of the bacteriophages. Alternatively or in addition to, the two or more bacteriophages may have other favorable properties, such as not infecting or lysing other bacterial types, such as C. granulosum. In another example, the host range data may be useful in determining two or more bacteriophages that infect, alone or in combination, all bacteria species within a group. For example, a combination of bacteriophage 13 and 58 can lyse all tested C. acnes strains and a combination of 13 and 58 with 87 can lyse all tested C. acnes and C. namnatense strains. A particularly beneficial combination of bacteriophages includes bacteriophages 2, 3, 7, 8, 9, 11, 67, 69, 85, 87, and 96.

Sequencing of DNA extracted from bacteriophage plaques, optionally including one or more DNA standards (as described elsewhere herein), may be performed to further characterize the bacteriophages and/or bacterial strains. An example protocol of preparing bacteriophage plaques for sequencing is as follows:

1. Prepare a phage stock in broth media:
1. Lyse a sensitive bacterial culture by adding phage during exponential-phase growth of the bacteria
2. Remove most bacterial biomass from the lysate by centrifuging at low speed and retaining the supernatant containing the phages
3. Filter the clarified lysate with a 0.2 μm filter to sterilize the phage stock
   2. Extract DNA from the phage stock:
1. Remove exogenous DNA by incubating phage stock with DNAse
2. Remove DNAse and digest phage capsids by incubating with Proteinase K and SDS
3. Purify DNA by loading onto a commercial silica-column DNA cleanup kit and washing with 80% Ethanol
3. Prepare a DNA library from the purified DNA using one of the commercially available DNA sequencing library preparation kits such as the Illumina Nextera XT or TruSeq kits.
4. Assay the quality of the DNA library and load onto the sequencing device.
5. Assemble the DNA sequencing reads into single contigs that represent the phage genome.

The sequencing data (e.g., sequence reads) may be further processed or analyzed, as described herein, e.g., to identify genetic elements of a bacteriophage associated with a host-range pattern. For example, one or more clustering analyses may be performed to determine sequence segments that are common to bacteriophages capable of infecting a given Cutibacterium strain. Accordingly, the determined sequence segments may be useful in identifying bacteriophages or bacteriophage types that are capable of lysing select Cutibacterium strains.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-73. (canceled)

74. A method of treating a skin condition of a subject, comprising:
(a) detecting a population of microorganisms in a skin sample obtained from said subject, wherein said detecting comprises performing shotgun metagenomic sequencing of nucleic acid molecules extracted from said population of microorganisms, thereby generating a set of sequence reads, and processing said set of sequence reads; and
(b) administering to said subject a bacteriophage or fungal virus formulation comprising at least one virus capable of lysing a microorganism of said population of microorganisms.

75. The method of claim 74, wherein said set of sequence reads consists of between 0.1 M-100 M sequence reads.

76. The method of claim 74, wherein said population of microorganisms comprises bacteria, fungi, mites, or viruses.

77. The method of claim 74, wherein said population of microorganisms comprises one or more microorganisms associated with acne.

78. The method of claim 74, wherein said population of microorganisms comprises one or more microorganisms associated with inflammation, redness, eczema, rosacea, enlarged hair follicle pore size, rough skin texture, increased trans-epidermal water loss, skin discoloration, disproportionate elasticity of a stratum corneum and underlying dermis, and aged skin.

79. The method of claim 74, wherein said population of microorganisms comprises a Cutibacterium acnes bacterium, a Staphylococcus aureus bacterium, a Corynebacterium bacterium, or a Malassezia yeast.

80. The method of claim 74, wherein said detecting further comprises (1) isolating deoxyribonucleic acid (DNA) from said skin sample and (2) removing human DNA from said isolated DNA in (1).

81. The method of claim 80, further comprising adding an internal DNA standard to said isolated DNA, wherein said internal DNA standard comprises synthetic DNA or naturally occurring DNA.

82. The method of claim 74, wherein said processing comprises (i) identifying one or more background sequence reads by shotgun genomic sequencing of deoxyribonucleic acid (DNA) isolated from a negative control sample and (ii) eliminating said one or more background sequence reads from said set of sequence reads.

83. The method of claim 74, wherein said processing comprises normalizing counts of said set of sequence reads to generate a set of normalized sequence reads.

84. The method of claim 83, wherein said processing comprises clustering of said set of normalized sequence reads.

85. The method of claim 83, wherein said processing comprises comparing said set of normalized sequence reads to a database.

86. The method of claim 85, wherein said database comprises sequences of a plurality of microorganisms isolated from other skin samples from other subjects.

87. The method of claim 85, wherein said database comprises sequences of mobile genetic elements, wherein said mobile genetic elements comprise one or more members of the group consisting of plasmids, prophages, and transposons.

88. The method of claim 74, wherein said bacteriophage or fungal virus formulation comprises a first bacteriophage and a second bacteriophage, wherein said first bacteriophage and said second bacteriophage are lytic bacteriophage.

89. The method of claim 88, wherein said first bacteriophage and said second bacteriophage have a different host range.

90. The method of claim 89, wherein said different host range comprises at least two C. acnes strains.

91. The method of claim 89, wherein said different host range comprises at least one C. acnes strain and at least one C. namnatense strain.

92. The method of claim 89, wherein said different host range does not comprise a C. granulosum strain.

93. The method of claim 88, wherein said first bacteriophage and second bacteriophage individually or collectively prevent resistance or regrowth of said microorganism in vitro.
